(19) United States
(12) Patent Application Publication
(10) Pub. No.: US 2021/0220504 A1
(43) Pub. Date: Jul. 22, 2021

(54) ULTRA HIGH EFFICIENCY ORGANIC GEL MICROBIAL AIR FILTRATION AND PRODUCTION SYSTEM

(71) Applicant: FILKIM FILTRE VE KIMYA SANAYI TICARET ANONIM SIRKETI, Istanbul (TR)

(72) Inventors: Burak Berk USTUNDAG, Istanbul (TR); Mehmet AKPINAR, Istanbul (TR)

(21) Appl. No.: 15/734,454

(22) PCT Filed: Jun. 10, 2019
(86) PCT No.: PCT/TR2019/050422

§ 371 Date: Dec. 2, 2020

(30) Foreign Application Priority Data
Jun. 4, 2018 (TR) 2018/07916

(51) Int. Cl. A61L 9/012; A61L 9/014; B01J 20/28; B01J 20/24; F24F 8/10; F24F 8/20; B01D 46/00; B01D 53/04

(52) U.S. Cl. CPC A61L 9/012; B01D 2257/91; B01J 20/28047; B01J 20/28045; B01J 20/2805; B01J 20/24; F24F 8/10; F24F 8/20; B01D 46/0036; B01D 53/0415; A61L 2209/14; A61L 2209/22; B01D 2253/20; B01D 2253/308; B01D 2253/304; B01D 2279/50; B01D 2259/4508; A61L 9/014

(57) ABSTRACT

Disclosed is an air cleaner filter containing an improved organic gel, wherein an ultra-high efficiency organic gel microbial air (UGMA) filter cartridge has a nano-porous texture on organic gel in spongy carcass diameters of which are randomly distributed, randomly distributed organic gel in spongy carcass as microbial retainer generated by a layer covering inner surfaces of a spongy carcass having randomly distributed pores at the grade selected in the range of 0.5 mm to 2.5 mm according to need, air inlet channels that allow air to be passed into sandwich structure UGMA filter cartridge, air flow channels such that microbial loads smaller than 0.1 pm are retained by pores and the air wipes the nano-pores by generating micro turbulence therein. Also disclosed is a production method for manufacturing the UGMA filter.

### TECHNICAL FIELD

The present invention in general relates to air filters and air cleaners and more particularly to an air cleaner filter containing an improved organic gel. The invention further includes a method for manufacturing the mentioned ultra-high efficiency organic gel microbial air filter (UGMA).

### STATE OF THE ART

Air filter systems are usually classified according to particle sizes they filter. Air filtering standards called as EPA, HEPA (High Efficiency Particulate Air Filter), ULPA (Ultra Low Penetration Air Filter), SULPA (Super Ultra Low Penetration Air Filter) therefore have criteria determining particle size that the filter is permeable and air flow resistance. However, there is no criteria related to microbial load of ambient air of which is filtered among the test criteria of those particle filter standards.

Particle filters operate as sieves. For instance, Department of Energy of United States of America identifies filters which are capable of removing 99.97% of particles measuring 0.3 μm as HEPA. Sieve apertures can be decreased since the manufacturing technology has advanced over the decades and ULPA type filters which are able remove 99.9995% of particles measuring 0.12 μm have been developed. However, pressure decrease generally occurs in the range of 150-300 Pascal at nominal air flow rate in HEPA filters based on sieve type particle filtration, whereas the pressure difference increasing when using ULPA class by decreasing sieve apertures increases the energy loss to much higher values. On the other hand, decreasing the filtration particle size below 0.3 μm is important in many fields including food, health and public health in community territories in order to filter airborne contagious common virus and bacteria including particularly influenza virus. Despite being an essential requirement, increasing the microbial efficiency could not gain as wide currency as HEPA filters except special requirements such as clean rooms because of its drawbacks based on costs particularly energy costs based on friction losses increasing due to their techniques by the methods based on decreasing sieve apertures with the short names ULPA, SULPA. There are endeavors to make improvements in order to overcome the energy efficiency problem and to increase microbial retention in sieve type particle filters. For example, patent with title “Hepa Filter” with publication No U.S. Pat. No. 6,428,610 B1 and publication date Aug. 6, 2002 and patent with title “Photocatalytic high-efficiency energy-saving air-filter net” with publication No CN204469537 U with publication date Jul. 19, 2015 are for increasing energy efficiency of particle filters. However, energy efficiency increasing mechanism of those are different from the subject matter UGMA filters. The one with publication No U.S. Pat. No. 6,428,610 B1 is based on electrostatic loading of nonwoven surface, and the one with publication No CN204469537 U is based on using photo-catalyst layer induced by ultraviolet light.

There are also methods electrostatically decreasing retained particle size without reducing energy efficiency in conventional particle removing filters such as disclosed in Patent document U.S. Pat. No. 7,258,729 B1 titled “Electronic bi-polar electrostatic air cleaner” with publication date Aug. 21, 2007. However, these kinds of methods use voltages at the degrees of kilovolts. High voltage restricts its areas of usage especially for personal uses and wet places.

Patent titled “Electrostatically enhanced HEPA filter” with publication No U.S. Pat. No. 4,781,736 A and publication date Nov. 1, 1988 relates to using high voltage electrodes at inlet of a sieve type filter component in order to increase particle removing efficiency by electrostatic ionization of particles in air at filter inlet in HEPA filters.

Patent titled “Gel Air Freshener and method of manufacturing same” with publication No US 2002/0039566 A1 and with publication date Apr. 4, 2002 relates to retaining different fragrances or agents such as deodorant inside a synthetic gel and dispensing them into surrounding air in time. However, usage of gel in patent titled “Air Filter for Removing Particulate Matter and Volatile Organic Compounds” with publication No US 2005/0132886 A1 and publication date Jun. 23, 2005 is exactly the opposite of UGMA. As disclosed in paragraph No [0018] of description of said patent, particles bigger than 0.3 μm are caught by gel and particles smaller than 0.3 μm are retained electrostatically.

### DESCRIPTION OF THE INVENTION

The invention disclosed herein, Ultra High Efficiency Organic-Gel Microbial Air Filter (UGMA), is based on breathing systems of organic gel surfaces in nature and on biomimetical design. The subject matter UGMA filter is based on usage of spongy structures as microbial retainer in multi-layer structures in sandwich form by being covered via organic-gel having high porosity at nanometer level. Among coarser particle and particle retainer layers, UGMA filters comprising component covered with organic-gel having high microbial and particle retention have high degree of retention against much smaller sized organisms relative to sieve apertures of sieve type filters such as HEPA and have lower energy loss. UGMA filter can be used on protective face masks, for air filtration of medical environments, and in places directly associated with human health such as food production facilities without any side effects since it does not contain any toxic or inorganic substances.

While UGMA operates by increasing organic gel coated surface wiping rate of air in random distributed air channels rather than sieve type particle filtration, total air friction is reduced, and the energy consumed is also reduced since the pressure difference between inlet and outlet surfaces.

In the state of the art, high voltage usage areas are restricted especially for personal uses and wet places.

Rather, retention of particles by electrostatically loading via high voltage is out of question in UGMA. In UGMA filtration, tri-hydroxyl and natural glucose bonds contained in organic gel and microbial loads at nanometer level are taken into Nano-porous organic gel structure with high efficiency.

Although gel usage at different ways on air conditioning and filtration are suggested, organic gel usage way on UGMA filter and resulting performance are different from all of them.

In contrast to the state of the art methods, in UGMA filter, the nano-porous structure in the air covered organic-gel progressing in randomly distributed spongy frame and the other microbial attractor organic materials in it also ensure the retention of particles and microbial particles especially smaller than 0.3 μm. In UGMA, an electrostatically treated surface or excitation via high voltage is not needed for retention of particles smaller than 0.3 μm. Furthermore, when UGMA filters are used in cycle air circulation, they can reach microbial load reducing rate at iterative half rate. Among the air filtration systems manufactured till now, none of them has successive reducing performance criteria in the manner of microbial load half per hour in cycle circulation except UGMA filtration.

Microbial activity is based on the assumption that microbial loads are carried by suspended agents in air and these agents are retained in particle filter. UGMA filter disclosed herein has microbial selectiveness unlike all air filters operating based on conventional particle retention. The subject matter UGMA type filter retains organisms having physical flexibility such as bacteria and viruses by a mechanism different from sieve type filters included in the standards. This mechanism is designed as biomimetic by inspiring from air breathing mechanism of living creatures in nature and is made to be applicable by industrially available materials. UGMA type air filter has structure in two different scales as macro and micro. In its macro scale, a distributed pore structure at mm levels, and in its micro scale, a porous structure at nanometer level are used together. Depending on air flow rate and stage order between filter layers, the pore structure at macro scale is obtained from a 1-100 ppi and typically around 20 ppi spongy material. Nano-porous structure is obtained from a gel material which varies according to the application target performance in the range of 50 nm-5000 nm and has special mixture. The gel texture covers all volumetric internal surfaces of the macro structure. The air cells occurred by this method also ensure that microbial loads are retained and depleted together with substance inside.

The present invention will be more apparent with the figures reference numbers of which are given below, and properties indicated in detailed description.

Performance comparisons between conventional, standard HEPA and ULPA filters and the UGMA filter disclosed herein can be seen in FIGS. 7, 8, 9 and 10. UGMA type filter cartridge used in those comparisons has three layered structure consisting of 200 gr/m² water pinning mask type felt coarse particle retainer at inlet surface of spongy framed structure having 6 mm thickness and 20 ppi pore density inner surfaces of which are gel covered, and 50 gr/m² nonwoven fabric at its outlet surface.

### DESCRIPTION OF THE FIGURES

FIG. 1 Three layered basic UGMA structure

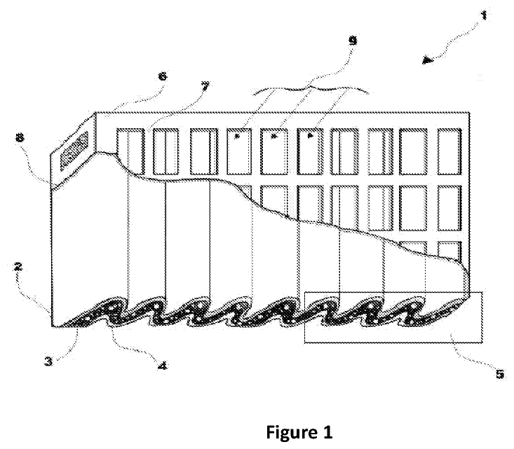

FIG. 2 Organic gel catching channels intra spongy skeleton and nano-porous organic gel nano-carcass design

FIG. 3 Organic-gel coating and UGMA production system

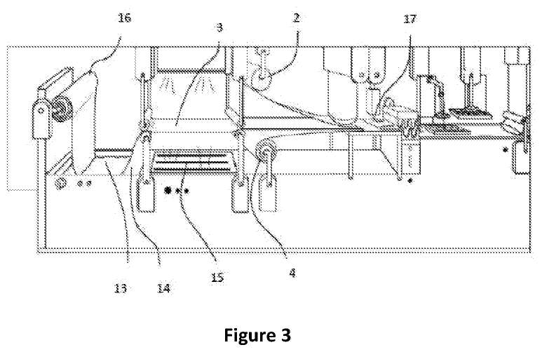

FIG. 4 In cycle UGMA air filtration system

FIG. 5 Multi-layer UGMA air filtration system at external air inlet

FIG. 6 UGMA filter in personal protection mask

FIG. 7 Variation of microbial load level in ambient air over time at closed cycle operation of UGMA filter and HEPA filters

FIG. 8 Variation of weighted average particle size in ambient air over time at closed cycle operation of UGMA, HEPA and ULPA filters

FIG. 9 Difference pressure decrease depending on air flow resistance on filter component according to weighted average particle size wherein ambient air of UGMA filter and sieve type air filters (HEPA/EPA/ULPA) will be reduced at closed cycle operation

FIG. 10 Variation of power consumption amount (PU: per unit) changed with usage time of UGMA Filter and Hepa filters and reduced to unit according to value at first operating moment over time

### DESCRIPTION OF THE REFERENCE NUMBERS

NO Part/Section Name
1 UGMA Filter cartridge
2 Coarse filter
3 Randomly distributed organic gel in spongy carcass
4 Nonwoven particle retainer layer
5 Undulation structure
6 Frame
7 Undulated structure retention lines on the frame
8 Film coating preventing the frame to absorb moisture
9 Air inlet channels
10 Air flow channels
11 Nano-porous texture
12 Tri-hydroxyl and glucose originated nutrition material
13 Heating and surface impregnation pool
14 Roller
15 Drying
16 Sponge not coated with gel
17 Ultrasonic stitching
18 Apparatus for in cycle air wiping
19 UGMA Filter cartridge replacement drawer
20 Releasing filtered air into ambient
21 Propeller for air suction
22 Optional electronic display and control unit
23 Strap and cable connection line
24 Optional illumination component
25 Outer air process unit for UGMA filtration
26 Multi-component series cartridge housing
27 Inlet air channel connection
28 Air inlet channel in personal protection mask filter
29 Organic gel filter
30 Big layered organic gel filter layers
31 Medium layered organic gel filter layers
32 Small layered organic gel filter layers

### DETAILED DESCRIPTION OF THE INVENTION

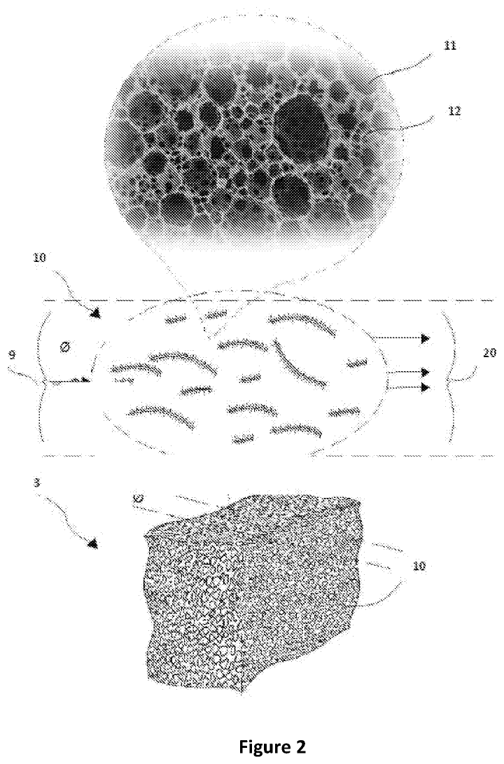

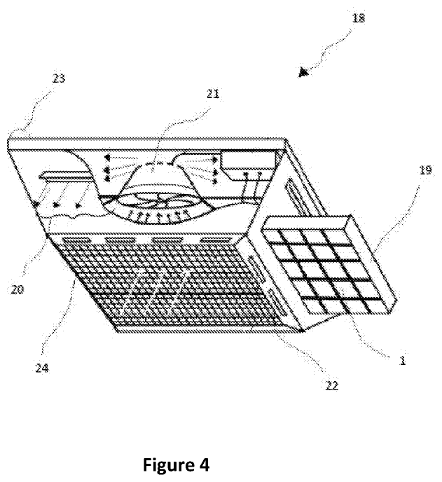

The most basic use of UGMA is only intra-volume air cycle as air purifier. In closed volume in cycle operation, a unit with propeller (21) for suction of air sucks air through the UGMA filter cartridge replacement drawer (19). In FIG. 4, pulled out state of UGMA filter cartridge replacement drawer (19) can be seen. The air entered from front face of apparatus for in cycle air wiping wherein UGMA filter cartridge replacement drawer (19) is installed flows through UGMA filter cartridge (1) through front face of at least 3 layered sandwich structured UGMA filter cartridge replacement drawer (19). In conventional air purifiers, particles are generally removed by sieve filters such as HEPA, ULPA and active carbon filters are used along with them. Methods and their derivatives such as ultraviolet light and microbial load reducing, enhancing particle retention by electrostatic ionization can take place in combination in conventional air purifying devices. Dissimilarly from all of those, in UGMA filtration, there is porous sponge or foam similar to lung mechanism, organic gel (3) inside randomly distributed spongy carcass, nano-porous texture (11) on organic gel covering randomly distributed channels, filter component retaining microbial and small particles. The microbial filter component consists of water pinning felt type coarse filter (2) on its inlet side and a nonwoven particle retainer layer (4) which retains the particles that may break on its outlet side. This surface expanding undulation structure (5) which is three layered sandwich structure, ensures that big particles in the incoming air and particles that can damage the middle layer are stopped in the first layer which is the coarse filter (2). The particles retained in this layer generally include particles which can also gravitationally gravitate. Randomly distributed spongy carcass which is the second layer has air channels in the organic gel (3) (FIG. 2).

The air flowing here is cleaned by a mechanism different from the sieve type filters. Air channels create turbulence by swaying to different directions in the form of fins. The air moving by swaying by this turbulence and comprising particle and microbial load wipes nano-porous texture (11) coated surfaces. FIG. 2 shows respectively starting from d=5 mm section sample scale, air flow channels (10) randomly distributed sponge aperture chosen according to air flow first with 100 micrometer and then 10 micrometer scale and nano-porous texture (11) on cross section sample and on section. Particles and microbial loads in the air wiping randomly distributed fins by turbulence fill with the tri-hydroxyl and glucose-based nutrition material (12) on the nano-porous texture (11). Therefore, average microbial load and particle amount reduction at closed cycle operation is increased according to air passed in proportion to duration. Because, as the filter component catches particles and microbial loads, the possibility of being retained is also increased together with the number of passes along with flowability of the remaining smaller ones. Another reason of this is that the tri-hydroxyl and glucose based nutrient material (12) on the nano-porous texture (11) are also randomly distributed and form the surface by being divided into as small components as the nm level. By means of this structure, flowing air does not encounter a constant pressure loss inversely proportional to sieve aperture such as in sieve type filters. Because in UGMA type filter, the average air aperture of the main filtration component is 100 times larger than the sieve type filters. By means of that, the energy loss is less than 50% since the flowing resistance is low and further, as filter is used, the significant increase in energy loss by being filled up in sieve type filters does not occur in UGMA type filters. The filtered air outlet from the gel filter texture coated spongy structure is applied to the nonwoven part retainer layer (4). This nonwoven particle retainer layer (4) prevents the organic gel (3) particles from mixing into ambient in the randomly distributed spongy carcass which may break from inside the filter and accumulated microbial particles in it. Furthermore, it ensures that medium size particles in proportion to paper pore are also retained.

The article prepared by Kanchan Maji, Sudip Dasgupta, Krishna Pramanik and Akalabya Bissoyi, titled “Preparation and Evaluation of Gelatin-Chitosan-Nanobioglass 3D Porous Scaffold for Bone Tissue Engineering” published in 2016 of journal named “International Journal of Biomaterials” of Hindawi publisher house relates to obtaining a gel carcass (scaffold) for usage of natural polymer-based bio-composite in bone tissue engineering. In UGMA filtration system, in contrast to that, the biomimetic effect in gel texture design is obtained by coating the porous hydro-gel carcass (Porous Scaffold) to a flexible spongy carrier structure in the form of film. Combining the naturally obtained bone gelatin with tri-hydroxyl in an aqueous medium and binding onto other organic substances especially microbial loads binding glucose constitute organic-gel carcass (scaffold) with Nano structural form seen in FIG. 2. This gel carcass has concave channel structures that can retain particles and microbial loads smaller than 0.1 μm. The air flow wipes the gel carcass that the spongy surface is covered with, and the loads it contains are retained by this carcass. According to the design of the gel, first level cavity diameters and the smaller cavity ratios within each of them, and the proportion of retainer material dispersed therein can be determined.

In FIG. 2, the gel covered spongy structure is shown in three different scales. The sponge structure at the bottom serves as the carrier of air channels at the level of randomly distributed millimeters. On its enlarged cross-sectional view, it is seen that the incoming air wipes the hydrogel film-coated surfaces. The microscope view of a cross-section taken from the surfaces here is given at the top. Accordingly, randomly distributed circular pores having different sized intertwined in the range of 10 nm-5000 nm cover the surface of the channels wiping air by directing, and the microbial loads and particles contained therein are retained by these pores.

Conventional air filters have low microbial achievement according to particle performance and also have short life. For example, in a conventional standard HEPA filter application replacement period of which is estimated for about 6 months, its life in terms of microbial retention ends between 15 to 45 days. The main reason for this is that the pressure increases in the remaining empty sieve apertures as the sieve intervals are filled in the sieve type filters, create nozzle effect and the microbial loads on the rigid particles hitting here are abraded and the free microbial loads can pass by flexibility.

UGMA filter increases the friction of the air to the surface to be filtered by turbulence by the passage of slimy gel material through cellular structured channels. Microbial loads in the air rubbing onto the surface are retained in nano-porous gel structure. Thus, it retains bacteria and viruses at the sizes that cannot be removed by conventional particle filters such as HEPA and ULPA, leading to lower pressure loss since its mechanism causes to less friction thereby resulting in a lower energy loss.

In cycle use of the UGMA filtration system, air wiping (flow distribution) is determined by minimum number of devices according to a procedure determined according to the indoor distribution of the microbial load production capacity of the ambient. Provided that this minimum ratio is ensured, half-life values per hour aimed with higher wiping values can be achieved due to mechanical conditions specific to places associated with the symmetrical appearance or installation access.

The production process is also effective in the microbial retention performance of the UGMA filter. Gel texture forming Nano pores should cover the spongy structure internal channels in the film structure at thickness as homogenously as possible. One of the methods developed for that is passing to cooling process at room temperature by compression after coating in hot gel pool. In terms of homogeneity, the hydrogel mixture is prepared first starting with the liquid substances. The mixture containing glycerin, water, gelatin and glucose is melted by being heated over 55° C. The sponge not coated with gel (16) to be the carrier of the air channels is immersed into the hydrogel-based mixture heating molten by being fed from roller and into the surface impregnation pool (13). By the volumetric gel discharge, all internal surfaces of the Nano pore texture are coated by being pulled to surface from the attractor roller (14). After drying (15) process, air channels, film coating (8) preventing the Nano-porous frame to absorb moisture, bio-carcass filter component are obtained. Bio-carcass filter component obtained in this manner, the material pulled from nonwoven particle retainer layer (4) and the felt type coarse filter (2) are pulled from material roller altogether and thus three-layered sandwich structure is obtained. Similarly, it is possible to obtain different multi-layer structures according to the requirements. Edge leakages of filtered air inside the cartridge are prevented by means of multi-layer sandwich structure, producing filter component at desired dimension by automatic cut, ultrasonic stitching (17) of the edges. The multi-layer filter structure is introduced into the undulation mold before being passed to the filter frame (6) for undulation on the air inlet direction. The undulated multilayer filter retainer is placed to the frame (6). Thereby, the retention performance according to energy consumption is increased, as the total surface is increased.

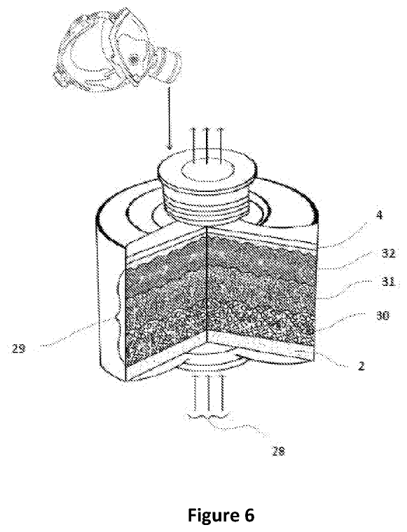

UGMA filters obtained in that manner can be used either in full cycle or partially together with the inlet air channel connection (27) having fresh air supply. the ambient is supplied with air from the structure of the multi component series cartridge housing (26) comprising a plurality of UGMA filter cartridges (1) because desired microbial filtering cannot be achieved from outer environment conditions by UGMA filter at one time in case of outer air supplying. As the personal protection mask air filters (FIG. 6), spongy air gap from inlet to outlet in personal protection mask filter big layered organic gel filter layers (30), spongy air gap from inlet to outlet in personal protection mask filter medium layered organic gel filter layers (31), spongy air gap from inlet to outlet in personal protection mask filter small layered organic gel filter layers (32) are used analogously in air intake and filtration through air inlet channel (28) of personal protection mask filter by means of natural flow such as breathing instead of drive by a motor.

In this case, the air passing from coarse filter (2) component to spongy air gap from inlet to outlet in personal protection mask filter big layered organic gel filter layers (30) passes to spongy air gap from inlet to outlet in personal protection mask filter small layered organic gel filter layers (32) which are retainer nonwoven filter layer at outlet by passing to spongy air gap from inlet to outlet in personal protection mask filter medium layered organic gel filter layers (31) from the spongy structure in the pore structure getting smaller from inlet to outlet as multi layered. The reason why the average pore size of hydro-gel coated spongy hollow structures in the intermediate layers varies from big to small is due to the need for uniformly distribution of the air resistance (pressure decrease) per layer in the filtration of the air taken at one time. In case of manufacturing it in equal pores, the wearing time of the spongy hollow layer in the first layer will be much shorter than the other layers.

In turbulent flow obtained by the fact that nano-porous gel covers macro air cells, retention of microbial loads in air wiping the surface is high. With nano-porous gel ingredients, retention along with nutrients, preservation of consistence in terms of natural viscosity and elasticity for a long time, thus increasing the effective life of the filter. In the sandwich structure, sensitive microbial gel filter (29) is prevented from being blocked by coarse particles by applying air passed through coarse filter (2) on organic gel filter (29) by means of felt (water pinning) retainer (200 gr/m²). Microbial load retaining particles are prevented from passing to clean ambient by deformation or hitting of smaller particles by means of nonwoven anti-microbial filter (50 gr/m²). Iteratively decreasing microbial load trend is obtained by means of the fact that the cycling rate per hour obtained as result of calculation made according to microbial production load and volume of the ambient is above the threshold cycling required for filter compensation. Total energy consumption is reduced significantly compared to sieve filtration systems of the same amount of equivalent retention due to the reduction of the air need intake from the external environment and due to filtration by the in cycle low pressure loss at larger amount. Air passing equivalent to multi cycle filtration ratio required for microbial retention is provided on passing over single route successively by the air introduced from outer ambient being passed through multi-layer sandwich structure. In the air filtration from the outer ambient, pressure and load distribution between layers are stabilized by gel filtration in texture measuring macro cell magnitudes reducing from large to small, thereby total filter system replacement life is lengthened by life narrow pass is brought to be equally distributed manner starting from inlet layer of outer ambient filter. Expanding the total surface area by undulation, thereby increasing the number of cells that the filter element can effectively use for the unit air flow and with lower internal flow rate retention efficiency, a higher total air flow rate is obtained. The continuity of the fresh air in internal ambient is ensured along with microbial filtration guaranty, by taking the intermittent air from at least two volumes at the outlet air intake, conducting the multi-turn reduction operations separately in each volume, and taking air into the internal ambient sequentially from the volumes obtained from the threshold filtration cycles. Nano-porous surfaces also increase the surface retention at different levels up to molecular scale in the emissions of harmful gases, especially organic mixed gases, and NBC (Nuclear, biological, chemical) agents in the air, thereby the physical decomposition of the carrier gases in the air, such as oxygen, from the mixed larger harmful molecules and particles is ensured. On the gas mask of the multi-layer UGMA filter, due to the increase of the wide spectrum retention effects of nano-porous and microcellular structure against harmful gas emission and broad-spectrum retention against NBC agents is ensured when transferring the air to air passage by human suction of air and cycling it, and thus, broad spectrum retention rather than conventional mask and ambient filter applications for conventional harmful gases or agents can be ensured. Instead of using special texture for sieve type and exposed gas or harmful chemical, since there is no major chemical interaction or screen mechanism of nano-porosity and micro-cell structure in mask or ambient filtration using the UGMA, its life is longer over 100% higher than conventional short-term filters. In UGMA filtration, Since the placement location and the number of the device are determined according to the physical distribution of total cycling capacity and productivity according to the balance criteria determined by the statistical measurement according to the microbial load productivity determined before the ambient filtration, since the UGMA filter element replacement period is determined according to iteratively continuously decreasing microbial load stability rate, and by using the countdown rate corresponding to that value as an automatic life estimator in the control unit of the UGMA filter air drive device, operating safety not exceeding the upper limit of the microbial load of the ambient is provided. By matching the device and filter production serial number in the ambient where the UGMA filter element is used as multiple, controlling of lifetime control in terms of all devices in the ambient and total ambient microbial filtration performance, measure validated UGMA filtering performance is ensured by blocking other filters or used filters of different character that do not exhibit the same performance. Changing the ratio of the substances added to the mixture to provide adhesion, viscosity and elasticity where the gel material provides high microbial filtration performance according to the operating temperature range of the usage environment, and by adding additional chemicals for high temperature ambient increasing the melting temperature, and for cold temperature ambient, lowering the freezing temperature, it is possible to provide aimed physical properties suitable for the ambient. Changing the ratio of the substances added to the mixture to provide adhesion, viscosity and elasticity where the gel material provides high microbial filtration performance according to the operating relative humidity range of the usage environment, and by adding additional chemicals for high temperature ambient increasing the melting temperature, and for cold temperature ambient, lowering the freezing temperature, it is possible to provide aimed physical properties suitable for the ambient. Since only the organic and non-toxic substances are used in the UGMA filter, it has no harmful effect on accidental ingestion. The UGMA filter can be subjected to standard disposal processes since it is preferred that its frame (6) and fasteners are manufactured from natural originated materials such as cardboard or wood, in terms of ease of waste disposal, including incineration against direct contact problems of the microbial loads it has retained during its use. Since the microbial retention of the UGMA air filter is above the conventional sieve filters, the high bacterial retention capability ensures that the shelf life of food is increased without increasing additive if present or without using any additive in food production environment or food chains where it is used. UGMA type filter lengthens staling time when used with cycle over filtration balance limit according to ambient in storing, cabin and shelf systems not completely isolated thanks to its microbial selectiveness and high energy efficiency, or, alternatively, instead of decreasing the conventional 4 degrees of bacterial growth by lowering the temperature, it provides further energy saving by the equivalent reproduction limitation of bacteria also at higher temperatures.

By means of these properties, the reduction of microbial contamination in the air in the production of food products such as dairy can increase the shelf life of the products by more than 100% without using additives. Analogously, in ambient sensitive such as hospitals and in crowded ambient such as public transportation and schools, since the microbial load in the air can be reduced including significant amounts of bacteria and viruses unlike other conventional filters with low energy loss, the infective and allergen effects can be greatly reduced.

In cases where HEPA type conventional sieve type air filter and UGMA filter are used in ambient where closed cycle air filtration is carried out, the variation of the microbial load level of the ambient with regard to time can be seen in Figure in proportional to the initial values. Accordingly, as in HEPA, while conventional sieve type filters reduce the level of microbial load in the ambient by converging to an asymptote decreasing as the sieve aperture gets smaller, the UGMA type filter brings the microbial load level to zero as its operating time increases. Because microbial loads can be transported to other ambient by hanging on particles larger than itself, which are usually suspended in air. The smallest structures are viruses and their sizes are below 0.1 microns. Bacteria are in the range of 0.15-0.30 microns. Molds measure up to 0.35 microns. Microbial agents have flexible structure due to their protein structure. Therefore, they can pass through pores smaller than themselves by means of high flow air. That is to say, since conventional filter structures consist of inorganic materials, most of the organic structure agents are able to pass through these pores. When the reduction level of microbial load ratio of HEPA filters over time is examined, it easily passes smaller structures (viruses, bacteria, yeast, mold) that are retained up to a certain level. In the case of UGMA filters, the microbial load level measured before is approximately halved per hour and comes to level of zero kbb.

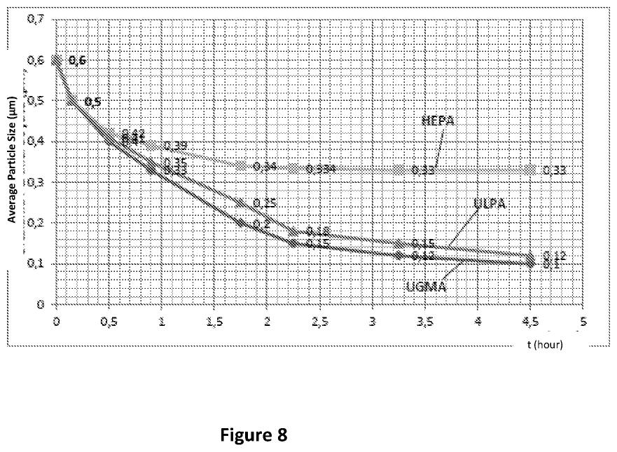

Variation of weighted average particle size in ambient air over time at closed cycle operation of UGMA, HEPA and ULPA filters can be seen in FIG. 8. These graphics were acquired by measuring the reduction levels of particles of various sizes in the first 5 hours in a closed ambient using HEPA filter, ULPA filter and UGMA filter. While the pore sizes of HEPA Filters are 0.33 microns, it can retain particles at 0.33 microns and larger. In smaller size ULPA filters, the particles of up to 0.12-micron size can be retained. UGMA Filters can retain particles of up to 0.1 micron in the first 4 hours by means of the gel filtration technology it contains. The reduction rate in the longer run surpasses the commercial conventional sieve type filters.

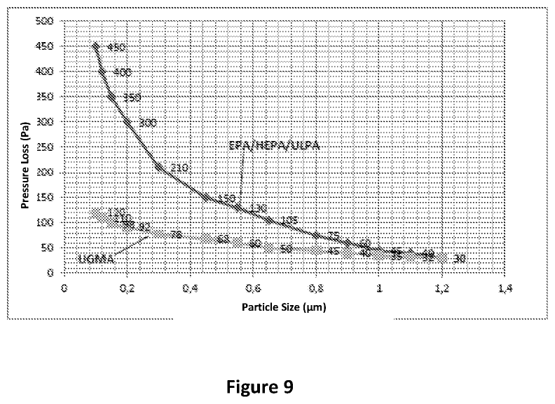

In sieve type filters, sieve apertures decrease from EPA to ULPA, but the pressure loss increases. Considering the parameters specified in EN 779 standard, where HEPA filter characteristics are classified, difference-pressure measurements vary according to the amount of air flow through the filter. As the air flow increases, the resistance increases thus difference-pressure rate will increase. HEPA filters can reach up to 450 pascals at air flow rate of 0.983 m3/s in the air test cabinet. The pore size must be reduced in order to retain smaller sized particles. In other words, it creates bigger difference-pressure ratio in order that smaller sized particles are retained. When the pressure generated in ULPA filters that provide up to 0.12-micron retention is at maximum 450 pascal level, this ratio in UGMA filters to reduce ambient air to the same particle size is maximum at 110 pascal level. FIG. 9 shows the difference pressure decrease depending on air flow resistance on filter component according to weighted average particle size wherein ambient air of UGMA filter and sieve type air filters (HEPA/EPA/ULPA) will be reduced at closed cycle operation. As the particle size increases, the pressure losses decrease. The pressure loss rate in MJH filters is between 30-120 pascals. This ratio is quite below those of conventional filters. Since its structure is different from other mechanisms, its air permeability is high.

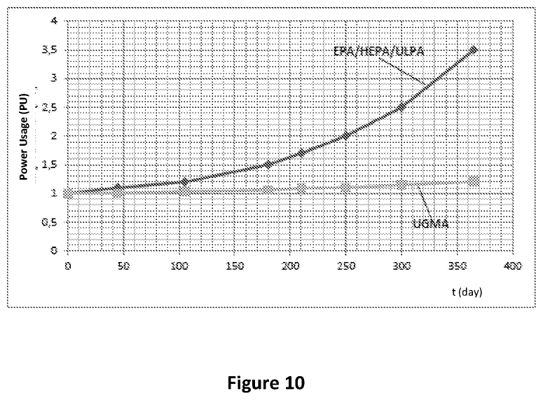

Since the particles retained in EPA, HEPA and ULPA filters clog the pore, the air flow that can pass to the opposite side decreases as the usage time increases and thus increasing the difference pressure loss. Therefore, as the number of days of usage increases, the power consumption increases, so that the daily energy consumption also increases. However, in UGMA filters, there cannot be any obstruction preventing the air flow as the usage time increases. As the number of days, the UGMA filter is used increases, Since the pressure loss in the main filter element does not change, and the changes due to coarse filter and non-woven filter layers are very small, its daily energy consumption change rate is significantly low compared to EPA/HEPA/ULPA filters. FIG. 10 shows the variation of power consumption amount (PU: per unit) changed with usage time of UGMA Filter and Hepa filters and reduced to unit according to value at first operating moment over time.

Furthermore, conventional filters filter the air from the outside and condition it and give it to the internal ambient. At least 6 times of change is carried out per hour. In other words, the air being heated or cooled and given to the environment is discharged out because of the number of changes. This leads to additional energy consumption as well as pressure loss. Since the inner ambient air in UGMA filter systems is filtered in cycle and brought back into ambient completely or with a higher ratio compared to HEPA filtration, a major part of the conditioned air stays within ambient and the energy consumption due to discharging and climatization of this decreases.

A significant characterization of Ultra High Efficiency Organic-Gel Microbial Air—UGMA Filter Cartridge is that; nano-porous texture (11) layer on randomly distributed organic gel (3) in spongy carcass is at average density selected in the range of 10 ppi to 50 ppi with respect to place to be used, and the air passing through air inlet channels (9) from a surface inside sandwich structured UGMA filter cartridge (1) of the randomly distributed organic gel (3) in spongy carcass in as microbial retainer generated by covering inner surfaces of a spongy carcass having randomly distributed pores wipes nano-pores by generating micro turbulences within air inlet channels (10) of spongy structure, and that it ensures microbial loads at sizes to be lower than 0.1 μm to be retained by pores. Thus, bacteria and viruses as small as not limited to the sieve aperture of sieve type filters such as HEPA, ULPA can be retained in the filter.

Another characteristic of UGMA filter cartridge (1) is that; since the air inlet apertures with size between 0.5 mm to 2.5 mm (10 ppi-50 ppi) of sponge pores in randomly distributed organic gel (3) in spongy carcass as microbial retainer is 1000 times bigger than the diameter of the smallest microbial load to be retained, air flow permeability is high and the pressure loss is low by wiping nano-porous texture (11) pores reduced down to nanometer grade. Thus, according to the increasing energy loss as the particle size to be retained decreases, as in sieve type filters such as EPA/HEPA/ULPA, UGMA filters provide energy savings regardless of the particle or microbial load size to be retained. FIG. 9 shows the difference pressure decrease depending on air flow resistance on filter component according to weighted average particle size wherein ambient air of sieve type air filters (EPA/HEPA/ULPA) will be reduced at closed cycle operation.

Another characteristic of the subject matter UGMA filter cartridge (1) is that; the pore number of the carcass in which the randomly distributed organic gel (3) in spongy carcass is located is 10 ppi-50 ppi (pores per inch). More preferably, 15-25 ppi porous material is used in commonly. The most commonly used 20 ppi pore density in a standard single-layer filter corresponds to an average pore aperture of 1.2 mm. Since the pore aperture of the carcass is in the grade of mm (0.5 mm-2.5 mm) and the air suspended substances do not reach up to this size, depending on the usage time as in sieve type filters, daily energy consumption change due to the increase in power consumption by clogging is at negligible levels compared to conventional HEPA and ULPA filters. (FIG. 10)

Another characteristic of the UGMA filter cartridge (1) is that; it comprises nano-porous texture (11) of hydrogel origin obtained from a mixture of organic gelatin, glycerin, water and glucose. Covering the inner surfaces of spongy structured filter component in the manner film; thus, a completely natural source microbial retainer air channel structure having no toxic effect with the porosity distribution determined by the proportion of the base materials in the mixture is obtained.

The nano-porous texture (11) herein is made to immovable by feeding into its mixture microbial load retained by glucose or other organic nutrients, whereas it can be disposed via optional carbonate.

The inner surface of the filter element contains small pores to retain the odor molecules in the incoming air, and the desired odor aroma can be mixed into the outlet air by natural odor aroma added to the hydrogel-glycerin mixture, i.e. to nano-porous texture (11), thus, it is possible to provide the desired odors while removing undesired odors in the air without the use of a deodorant suppressant chemical. Moreover, organic coloring agent can be added to the nano-porous texture (11) contained in the subject matter UGMA filter cartridge (1).

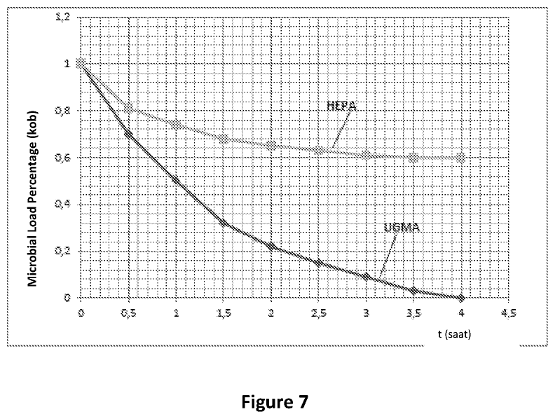

A further characteristic of the subject matter UGMA filter cartridge (1) is that; it can retain particle and microbial loads which are getting smaller in size by converging to zero grade as the application time is increased since retention possibility of the randomly distributed nano-porous texture (11) on inner surface of randomly distributed organic gel (3) in spongy carcass a certain sized particle or microorganism is proportioned to total air volume wiped if continuous closed cycle ambient air circulation with the device (18) for in cycle air wiping inside the same ambient is provided; thus, although the air filtration particle size cannot be reduced to below the sieve aperture in sieve type filters such as HEPA, it is also possible to reduce smaller microbial loads in the ambient as the system run time increases in closed cycle in UGMA filters. (shown in FIG. 7-FIG. 8)

A further characteristic of the UGMA filter cartridge (1) is that; the energy loss of larger particles degrading nano-organic randomly distributed organic gel (3) in spongy carcass by the felt type coarse filter (2) attached to its inlet is retained in low inlet layer relatively.

A further characteristic of the UGMA filter cartridge (1) is that; the particles that can break from nonwoven particle retainer layer (4) and randomly distributed organic gel (3) texture in spongy carcass are retained, thus the particles having microbial load in its body are prevented from mixing into the ambient.

The production method of Ultra high efficiency microbial gel—UGMA filter cartridge (1), characterized in that; the sponge not coated with gel (16) is immersed into the heat and surface impregnation pool (13) of the nano-porous texture (11) that is hydrogel-based mixture molten by being heated over 55° C. and then it covers all inner surfaces of nano-porous texture (11) by being pulled from roller (14) and comprises drying (15) process steps.

Another characteristic of the production method of UGMA filter cartridge (1) is that; the material and felt type coarse filter (2) materials pulled from nonwoven particle retainer layer (4) are pulled from the roller together and UGMA filter cartridge (1) of desired size is produced by automatic cutting, the edge leakages of filtered air inside the cartridge are prevented by means of ultrasonic stitching (17) and this three layered structure is placed on retainer frame (6) by means of undulated structure retainer lines (7) with undulation structure (5) in the direction of air inlet, thereby, the retention performance according to energy consumption is increased, as the total surface is increased.

A further significant characteristic of the subject matter UGMA filter cartridge (1) is that; glycerin, water, sugar and other additives added to the hydrogel material covering the spongy texture—nano-porous texture (11) and the porosity size distribution in the nanometer-micrometer scale range can be controlled. Thus, it is possible to perform performance optimization in accordance with the target between the activity period, life and minimum particle level according to the place to be implemented.

Another characteristic of the UGMA filter cartridge (1) is that; the filter surface area within the undulating structure (5) and the volume of the UGMA filter cartridge (1) is increased, thereby the differential pressure-based energy loss is reduced while increasing the air flow rate.

A further characteristic of the UGMA filter cartridge (1) is that; instead of microbial and particle load reduction by multi-turn cycle in the free air flow with respiration, spongy layers of different pore sizes from larger to smaller; spongy air gap from inlet to outlet in personal protection mask filter big layered organic gel filter layers (30), medium layered organic gel filter layers (31), small layered organic gel filter layers (32) are used. Thus, in applications such as a protective mask, air filtration at the desired level in a single pass is ensured.

A significant characteristic of the UGMA filter cartridge (1) is that; it comprises an outer air process unit (25) for UGMA filtration at the outside air inlet when fresh external air is required to be added to ambient. As an alternative to microbial and particle load reduction by multi-turn cycle in a separate division, the spongy layer inner surface air channels of which is coated with gel is ensured to be used more than once together with multi element serial cartridge housing (26). Thus, beside maintaining the air microbial load of which is reduced in inner ambient by single turn air passing at aimed cleaning level, also equal pressure decrease is ensured between the layers according to the pore aperture change.

An important characteristic of the outer air process unit (25) for said UGMA filtration is that; pore apertures and thickness of the sponge layers from the air inlet to the outlet are sorted by being sized such that the pressure decrease distribution per layer is kept balanced at equal air flow, thus the compound life of the filter component is lengthened by avoiding shortening due to further wear of the input layer.

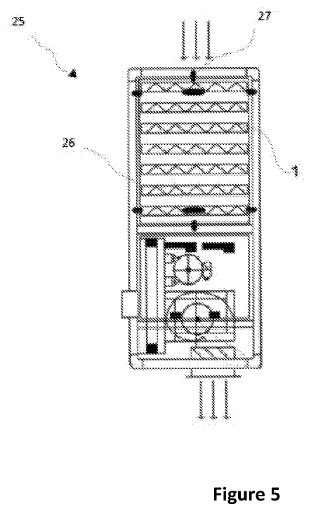

Pore intervals are in the range of 8 ppi to 10 ppi according to the total number of layers at the first inlet. If the number of layers is more than 6, it starts with a maximum of 12 ppi. The ppi values on each layer increase from inlet to outlet. In the last layer, the pore density is between 20-25 ppi according to the upper limit of air flow rate of the system. When intermediate pore layers are not available in standard production, also a reduction method in groups can be used. For example, 9 layers can be made in the manner of 10 ppi, 10 ppi, 10 ppi, 10 ppi, 10 ppi, 20 ppi, 20 ppi, 20 ppi, 20 ppi from inlet to outlet respectively. In case that the pore diameters are sorted from large to small in multi-layer outer air inlet filter application, according to the equal pore aperture situation, the wear earlier than average period as a result of the pressure imbalance in the input layer is prevented. Pore aperture decrease amounts are determined according to the number of layers. The number of layers is maximum 12. The case when the number of layers at the outer inlet is 12, is the case equal to the number of intra cycles per hour in the standard. In the case with less than 12 inlet layers, the remaining cycles are reduced in the closed cycle inside. The layers in FIG. 5 represent the inlet from the outer unit.

1. An ultra-high efficiency organic gel microbial air (UGMA) filter cartridge comprising:
randomly distributed organic gel in spongy carcass as microbial retainer generated by a layer covering inner surfaces of a spongy carcass having randomly distributed pores at the grade selected in the range of 0.5 mm to 2.5 mm;
a nano-porous texture on organic gel in spongy carcass diameters of which are randomly distributed;
air inlet channels that allow air to be passed into sandwich structure UGMA filter cartridge;
air flow channels such that microbial loads smaller than 0.1 μm are retained by pores and air wipes nano-pores by generating micro turbulence therein.

2. An ultra-high efficiency organic gel microbial air filter cartridge according to claim 1, comprising organic gel in randomly distributed spongy carcass having air flow apertures at mm grade of sponge pores thereon such as microbial retainer which is 1000 times larger than the diameter of the smallest microbial load (particularly particles and microbial particles less than 0.3 μm) to be retained.

3. An ultra-high efficiency organic gel microbial air filter cartridge according to claim 1, comprising nano-porous texture of hydrogel origin obtained from a mixture of organic gelatin, glycerin, water and glucose.

4. An ultra-high efficiency organic gel microbial air filter cartridge according to claim 1, comprising nano-porous texture containing carbonate.

5. An ultra-high efficiency organic gel microbial air filter cartridge according to claim 1, wherein the inner surface of the filter component contains small pores to retain odor molecules in incoming air.

6. An ultra-high efficiency organic gel microbial air filter cartridge according to claim 1, comprising nano-porous texture having a hydrogel-glycerin mixture containing a natural odor aroma.

7. An ultra-high efficiency organic gel microbial air filter cartridge according to claim 1, comprising nano-porous texture containing nutrients of tri-hydroxyl and glucose origin thereon.

8. An ultra-high efficiency organic gel microbial air filter cartridge according to claim 1, wherein, in order to ensure retention of particle and microbial loads which are getting smaller in size by converging to zero grade as the application time is increased ensuring that retention possibility of a certain sized particle or microorganism is proportioned to total air volume wiped, it comprises randomly distributed nano-porous texture on inner surface of randomly distributed organic gel in spongy carcass if continuous closed cycle ambient air circulation with the device for in cycle air wiping inside the same ambient is provided.

9. An ultra-high efficiency organic gel microbial air filter cartridge according to claim 1, comprising felt type coarse filter retaining the energy loss of larger particles at inlet relatively degrading nano-organic randomly distributed organic gel in spongy carcass.

10. An ultra-high efficiency organic gel microbial air filter cartridge according to claim 1, comprising a nonwoven particle retainer layer at the outlet of the filter cartridge, which ensures retention of particles that may break from organic gel texture in randomly distributed spongy carcass.

11. An ultra-high efficiency organic gel microbial air filter cartridge according to claim 1, comprising nano-porous texture containing organic colorant.

12. An ultra-high efficiency organic gel microbial air filter cartridge (1) according to claim 1, wherein the pore number of the carcass in which the randomly distributed organic gel in spongy carcass is located is 10 ppi to 50 ppi.

13. An ultra-high efficiency organic gel microbial air filter cartridge according to claim 1, comprising spongy layers of different pore sizes from larger to smaller instead of microbial and particle load reduction by multi-turn cycle in the free air flow with respiration; spongy air gap from inlet to outlet in personal protection mask filter big layered organic gel filter layers, medium layered organic gel filter layers, small layered organic gel filter layers.

14. An ultra-high efficiency organic gel microbial air filter cartridge according to claim 1, comprising an outer air process unit for UGMA filtration at the outside air inlet when fresh external air is required to be added to ambient.

15. The outer air process unit for ultra-high efficiency organic gel microbial air filtration according to claim 14, wherein pore apertures of the sponge structure layers from the air inlet to the outlet are in the range of 8 ppi to 15 ppi at first inlet.

16. The outer air process unit for ultra-high efficiency organic gel microbial air filtration according to claim 14, wherein pore apertures of the sponge structure layers from the air inlet to the outlet are in the range of 20 ppi to 25 ppi at last layer.

17. An ultra-high efficiency organic gel microbial air filter cartridge according to claim 1, wherein, as an alternative to microbial and particle load reduction by multi-turn cycle, the spongy layer inner surface air channels of which are coated with gel ensuring usage of more than once comprises multi component serial cartridge housing.

18. The production method of ultra-high efficiency microbial gel-filter cartridge, wherein the sponge not coated with gel is immersed into the heat and surface impregnation pool of the nano-porous texture that is hydrogel-based mixture molten by being heated over 55° C. and then it covers all inner surfaces of nano-porous texture by being pulled from roller and comprises drying process steps.

19. An ultra-high efficiency organic gel microbial air filter cartridge according to claim 18, comprising the process steps of: pulling material and felt type coarse filter materials pulled from nonwoven particle retainer layer from roller together and producing UGMA filter cartridge of desired size by automatic cutting, preventing edge leakages of filtered air inside the cartridge by means of ultrasonic stitching and placing this three layered structure on retainer frame with undulation structure in the direction of air inlet.
